(19) United States
(12) Patent Application Publication
(10) Pub. No.: US 2023/0142726 A1
(43) Pub. Date: May 11, 2023

(54) ASSESSMENT TEMPLATE AND PLAN GENERATION FOR ACTIVITY MANAGEMENT

(71) Applicant: Rational Alliance, Inc., Las Vegas, NV (US)

(72) Inventors: Robert Atlas, Las Vegas, NV (US); Robert Clymer, Las Vegas, NV (US)

(21) Appl. No.: 17/540,147

(22) Filed: Dec. 1, 2021

Related U.S. Application Data
(63) Continuation-in-part of application No. 17/524,014, filed on Nov. 11, 2021.

(51) Int. Cl. G16H 40/67; G16H 10/60

(52) U.S. Cl. CPC G16H 40/67; G16H 10/60

(57) ABSTRACT

One or more techniques and/or systems are disclosed for life management topic (LMT) assessment template and plan generation, wherein LMT domain inputs are received via a user interface. Components of an LMT assessment template are defined using the received LMT domain inputs. The LMT assessment template is output based at least on the defined components, wherein the LMT assessment template is a relational template having cross-referencing between one or more of the defined components to define a customizable assessment. One or more LMT plans are generated using the LMT assessment template and based on the customizable assessment.

### CROSS-REFERENCE TO RELATED APPLICATION

This is a Continuation-in-Part of Application Serial No. 17/524,014, entitled Assessment Template and Plan Generation for Activity Management, filed Nov. 11, 2021. The disclosure of the prior application is hereby incorporated by reference herein in its entirety.

### BACKGROUND

Life management systems, such as life management software designed to help aging seniors, typically present a user with a static assessment workflow. For example, in the case of senior management systems, the assessment workflow allows a user to note which activities of daily living (ADLs) the senior can perform on their own and with which ones the senior will need assistance. The assessment allows the user to answer general questions and select fixed options that may result in the creation of a care plan for the senior that is undifferentiated since the plan lacks the ability to incorporate more detailed assessment information about the particular senior. That is, the care plan consists of high-level questions with fixed care elements. As such, these life management systems are limited in the ability to allow system administrators to change or tailor the elements based on different applications or environments, unable to give users the ability to automatically create differentiated care plans based on the input of detailed assessment information and option selection, and do not provide a feedback mechanism whereby manual and automated monitoring of the care plan execution can inform the user of the need to perform a reassessment as well as inform the system administrator of the need to tune the assessment elements and logic for subsequent improved user interactions with the assessment.

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

One or more techniques and systems described herein can be utilized for life management topic (LMT) assessment template and plan generation, as well as activity management. For example, systems and methods of generating LMT assessment templates and plans described herein, can utilize a data driven approach to more effectively and efficiently configure LMT assessment templates that generate plans with feedback from plan execution.

In one implementation, a computerized method for generating life management topic (LMT) assessment templates includes receiving LMT domain inputs via a user interface and defining components of an LMT assessment template using the received LMT domain inputs. The computerized method further includes outputting the LMT assessment template based at least on the defined components, wherein the LMT assessment template is a relational template having cross-referencing between one or more of the defined components to define a customizable assessment. The computerized method also includes generating one or more LMT plans using the LMT assessment template and based on the customizable assessment.

To the accomplishment of the foregoing and related ends, the following description and annexed drawings set forth certain illustrative aspects and implementations. These are indicative of but a few of the various ways in which one or more aspects may be employed. Other aspects, advantages and novel features of the disclosure will become apparent from the following detailed description when considered in conjunction with the annexed drawings.

### BRIEF DESCRIPTION OF THE DRAWINGS

FIG. 1 is a block diagram illustrating one implementation of a life management topic (LMT) assessment template generator.

FIG. 2 is a block diagram illustrating one implementation of an LMT system.

FIG. 3 is a flowchart of a process flow illustrating operations involved in LMT assessment template and plan generation and execution according to one implementation.

FIG. 4 is a block diagram illustrating a data structure according to one implementation.

FIG. 5 illustrates an interface according to one implementation.

FIG. 6 illustrates another interface according to one implementation.

FIG. 7 illustrates another interface according to one implementation.

FIG. 8 illustrates another interface according to one implementation.

FIG. 9 illustrates another interface according to one implementation.

FIG. 10 illustrates a portion of an interface with options according to one implementation.

FIG. 11 illustrates cross-referencing according to one implementation.

FIG. 12 illustrates an example implementation of a method for defining and updating an LMT assessment template and plan.

FIG. 13 is a block diagram of an example computing environment suitable for implementing various examples of LMT planning and management.

### DETAILED DESCRIPTION

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

The methods and systems disclosed herein, for example, may be suitable for use in life management planning and execution of life management plans for many different applications. For example, assessment templates and plans are dynamically created and managed, with feedback being used to confirm the performance of activities or other actions associated with one or more generated assessment templates and/or generated plans. It should be appreciated that the herein described examples can be used in different settings or environments, such as for different types of life management functions or care giving (e.g., elderly, sick, rehabilitation patients, etc.). The examples given herein are thus merely for illustration.

In some implementations, the assessment workflow is extended by allowing for the creation of assessment templates. For example, an assessment template is created using inputs from a system administrator. The system maintains information from the inputs that defines a logic tree within each assessment template, along with resulting plan elements (e.g., care plan elements) created based on the user’s path through the assessment template logic trees. By employing assessment templates of the various examples described herein, assessments can be used to solve many different and novel life management problems, such as determining household management activities, financial planning activities, targeted activities, etc. In some examples, techniques described herein can be configured (e.g., software reused) to solve different life management problems by providing different dynamically configurable assessment templates. As such, different provisioners of the systems can differentiate offerings by providing different assessments configured or designed for particular users or use cases. In some examples, users can be offered different templates based on an individual’s particular goals, characteristics, and selection process.

In one or more implementations, along with the logic tree information, assessment templates also contain the information for users to assess the need for clustered activities. An example of a clustered activity is a medication list for the senior. As such, an assessment template can be defined to present the user with the ability to, for example, log which medicines a senior should take and include the times of day (e.g. with breakfast, before bed, etc.) for taking the medicines, instructions, reorder information, etc. This information is used in various examples to create elements of a care plan. In one example, within the care plan’s recurring activities, a single entry for each relevant time of day is created. For example, with recurring activities such as one with breakfast and one before bed, an entry for each with relevant instructions detailing all the medications to be taken at each time respectively is generated. Thus, various examples provide templated life management assessments.

In some implementations, recurring activities management is also provided. For example, as described herein, the care plan includes a list of recurring activities that should be performed daily, weekly, etc. The care plan results in the generation of a checklist each day that one or more caregivers can use as a guide to make sure each activity is performed. Some examples provide a management process for the checklists, such that each individual activity on each checklist can be marked as having been performed, partially performed, or not performed as expected. For any particular activity, this marking can be performed manually (via a user interface) by a caregiver assisting the senior or can be automated through an interfaced data upload from external systems (e.g., monitors, cameras, user activity devices, care robots, digital therapeutic devices, etc.). For example, if the senior has a checklist activity to go out for a walk on a given day, a geo-locator on the senior’s mobile phone can have the information needed to mark the walk activity as having been performed. Additionally, the accelerometer on the mobile phone can track if the senior fell during the walk and mark the walk activity as not performed as expected along with the relevant reason why the walk was not performed as expected (e.g., senior fell).

In one example, for each activity partially performed, not performed as expected, or not marked at all (after a deadline duration has been reached), the system can send an alert to other users of the system. Since not all failures to perform a checklist activity need an alert, whether or not the alert occurs for any particular activity can be defined as part of the assessment template.

Thus, improvements to a life management system are provided that can result in the generation and execution of care plans being faster, less costly, and more accurate. That is, more focused or customized care plans can be generated with more accurate tracking of activities performed. In this manner, when a processor is programmed to perform the operations described herein, the processor is used in an unconventional way that allows for more efficient and accurate care plan generation and execution, which results in an improved user (e.g., customer) experience.

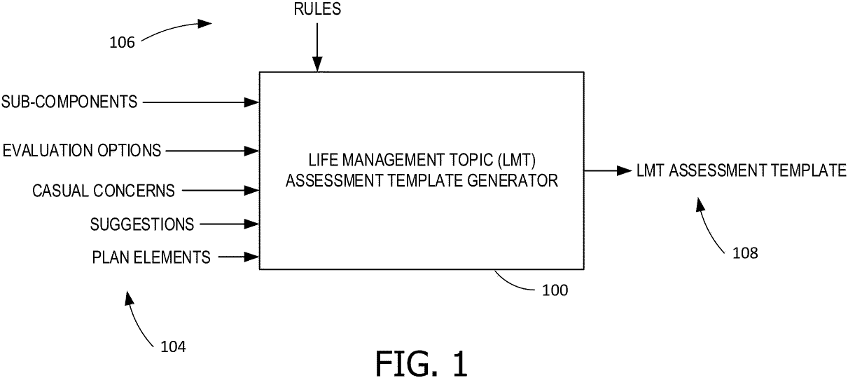

As one example, an LMT assessment template generator 100 is illustrated in FIG. 1. The LMT assessment template generator 100 is configured to receive one or more inputs, such as different components, elements, criteria or variables 104, which can be any inputs that affect the assessment template, such as the care plan to be generated therefrom. In the illustrated example, the variables 104 include sub-components, evaluation options, causal concerns, suggestions, and care plan elements. However, it should be appreciated that the illustrated variables 104 are merely for example, and other variables can be used by the LMT assessment template generator 100. That is, the LMT assessment template generator 100 can be configured to generate different types of assessment templates, such as for activities of daily living, household management, quality of life, etc.

With respect to the variables 104 that are arranged in a logic tree data structure in some examples, following are one or more factors or characteristics for each:

1. Sub-components - general elements for assessment that define different categories of activities, actions, etc. For example, sub-components in one example for elder care include: eating and drinking; continence and toilet use; transferring and mobility; dressing; bathing and grooming; and taking medications.

2. Evaluation options - define sufficiency levels for each of the sub-components. That is, the evaluation options include selectable options based on measurable or observable competency or completion of actions. For example, evaluation options in one example for elder care include: sell sufficient; needs some assistance; and needs full assistance. Thus, evaluation options are defined for each sub-component.

3. Causal concerns - define concerns relating to activities within each of the subcomponents. In some examples, the causal concerns define possible areas of concern for each of the subcomponents, which can include different concerns for each (or some of the same concerns). For example, evaluation options in one example for elder care include: (1) for eating and drinking - trouble swallowing, trouble keeping food down, not able to use utensils properly (stroke, Parkinson’s, physical limitation, etc.), cognitive issues, and dehydration; (2) continence and toilet use - physical inability to hold in urine or bowel, cannot get to the toilet easily, cannot use the toilet, and cognitive issues; (3) transferring and mobility - excess fear or history of falling, sedentary lifestyle and obesity, balance impairment, medical mobility limitations, and cognitive issues ; (4) dressing - limited range of motion or coordination, trouble choosing or wearing appropriate clothing, trouble with jewelry or accessories, safety issues, and cognitive issues; (5) bathing and grooming - depression, fear of falling in shower or tub, pain or physical limitations, and cognitive issues; and (6) taking medications - trouble swallowing, cannot apply non-oral medications, trouble opening bottles, fear of side effects, addiction or trust the medication is needed, and cognitive issues. Thus, causal concerns are defined for each evaluation option of each sub-component

4. Suggestions - define suggestions to address or are related to each of the causal concerns, which can include different suggestions for each (or some of the same suggestions). For example, suggestion options in one example for elder care include evaluations, examinations or check-ups, further monitoring, avoidance (e.g., avoiding certain activities or actions), obtaining/requesting implements, items, medications, etc., interactions, verifications, modifications (e.g., modified schedules, activities, etc.), arranging items, installing items, removing items, alternate items, ensuring actions, installing detector/monitor, joining groups, physical or mental assistance, and organizing. Thus, suggestions are defined for each causal concern.

5. Care Plan elements - default LMT care plan elements, such as “to dos” for the overseer and recurring activities and instructions for caregivers. It should be noted that in some examples, default alerting rules are defined for failed recurring activities. Thus, care plan elements are defined for each suggestion.

It should be noted that while in various examples suggestions with elements are output, the user is able to accept or decline/ignore the suggestions, which results in the instantiation of the care plan elements. That is, one or more user inputs or responses leads to the instantiation of the care plan elements.

In various examples, a data driven approach to template generation allows for dynamic creation of templates with one or more user definitions. For example, a relational assessment template is defined by a data structure that drives the assessment template creation, such as a data driven checklist for elder care as described in more detail herein.

In operation, the LMT assessment template generator 100 receives the variables 104 and additional inputs 106 (e.g., rules that determine which users or assessors have access to an LMT assessment template), processes the variables 104 and the additional inputs 106, and generates as an output, an LMT assessment template 108 for a life management or care plan. The LMT assessment template generator 100 allows for dynamically generating many different types of LMT assessment templates, wherein one or more instances of the LMT assessment templates can then be run on a user device, which allows interaction thereof with, for example, a user interface.

In some examples, the LMT assessment template generator 100 can be implemented in software, hardware, or a combination thereof. For example, a series of software packages implementing one or more aspects of the LMT assessment template generator 100 can be provided. In this example, the LMT assessment template generator 100 is configured to generate one or more LMT assessment templates that are used to create care plan(s) with activities and tasks for caregivers and others involved in, for example, a life management environment (e.g., elder care, sick care, etc.). In one example, a checklist that can be followed, i.e., a daily care plan, is generated. A responsible individual receives feedback, such as indicating that an activity or task is not completed and/or is completed. It should be appreciated that by using the LMT assessment template generator 100, the care plan is thereby made dynamic. That is, the care plan is not static, for example, as to what the caregiver should be doing with the responsible individual (sometimes referred to as an “overseer” for the person for whom care is being provided) and can include, for example, adding/changing tasks.

It should be appreciated that the LMT assessment template generator 100 is not limited to generating daily care type assessment templates, but can be used to generate assessment templates for other applications. In some examples, a plurality of assessment-based modules is provided to generate different types of assessment templates and corresponding plans, such as care plans. However, in various examples, the different plans can be generated from the same base assessment templates. For example, elder care plans, sick care plans, rehabilitation plans, home management plans, end of life plans, etc. are generated using the LMT assessment template generator 100 (or other template generators configured as described herein) and based on different defined variables 104.

In some examples, the LMT assessment template generator 100 is configured to generate preprogramed, predefined, or prepopulated assessment templates. In one example, empirical, experimental or simulation data is used to train or configure the LMT assessment template generator 100, and then the variables 104 and optionally the additional inputs 106 are processed to generate one or more assessment templates for a particular application. In some examples, machine learning is used to train or configure the LMT assessment template generator 100 based on a training data from simulations or feedback received from previously generated assessment templates to converge to more relevant templates and corresponding plan (e.g., care plan). In some examples, artificial intelligence (AI) is used as part of the training of and/or processing by the LMT assessment template generator 100 to generate improved assessment templates.

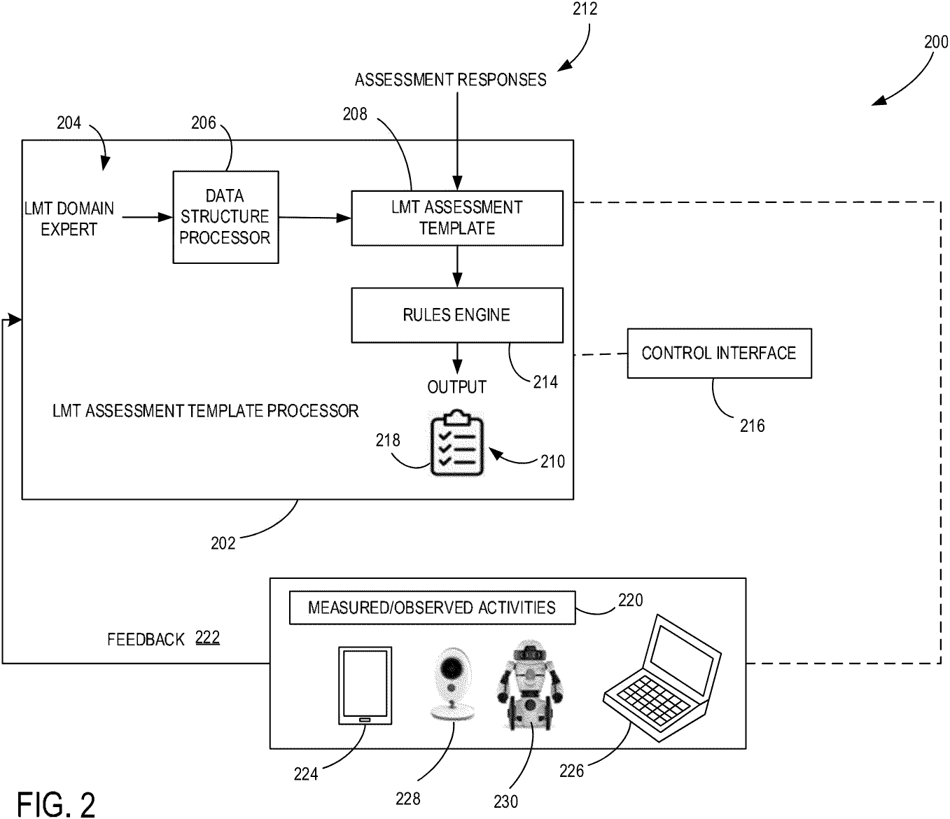

One particular implementation includes an LMT system 200 as illustrated in FIG. 2. In some examples, the LMT system 200 is implemented as part of or includes the LMT assessment template generator 100. The LMT system 200 in one example is a processing machine that can be used in combination with one or more other systems (e.g., monitoring systems) to generate LMT assessment templates, LMT plans (e.g., care plans), and facilitate executing the LMT plans. More particularly, the LMT system 200 includes an LMT assessment template processor 202 that is configured as a processing engine that performs assessment template generation using input data 204, which can include the variables 104 and optionally the additional inputs 106, illustrated as LMT domain expert data or inputs. It should be noted that the input data 204 can include different types of data configured in different ways corresponding to different types of LMTs, different LMT applications to be performed, etc. It should also be noted that the examples described in the present disclosure can be applied to different types of data, including non-LMT data. In some examples, the input data 204 is obtained via a user interface having selectable element or fields. As such, in various examples, manual entry of input data (e.g., LMT domain expert data) into a spreadsheet expert system is not performed.

In operation, the LMT assessment template processor 202 has access to the input data 204, such as the different types of variables 104 and different elements received as inputs for an LMT application to be performed (e.g., as part of an elder care assessment and care plan). It should be appreciated that the LMT assessment template processor 202 is configured to perform assessment template generation in a wide variety of application domains. For example, the implementations of the present disclosure provide for assessment template generation and execution in an LMT domain, but various implementations can be used in other domains, such as with other domain experts providing input data.

In the illustrated example, the input data 204 includes variables defining one or more assessment elements or components, wherein the LMT assessment template processor 202 processes the input data 204 using a data structure processor 206 as described in more detail herein. In some examples, the data structure processor 206 processes the input data 204 using one or more algorithms or tree structure processing engines that use metadata corresponding to the input data 204 to create an LMT assessment template 208. That is, the data structure processor 206 generates relational assessment templates wherein a data structure (e.g., a data tree structure) is used for generating and driving the generation of the LMT assessment template 208. As such, the input data 204 is used to determine or define assessment elements (e.g., an assessment template structure) for a desired or required application based on the received input data 204 and generate the corresponding LMT assessment template 208.

In the implementation illustrated in FIG. 2, the LMT assessment template processor 202 can also perform processing using one or more assessment responses 212 to generate an output 210. For example, using one or more assessment answers (in response to assessment input requirements or queries), the LMT assessment template processor 202 applies a data-driven rules engine 214 to construct the output 210, which in various examples are identified tasks for the care plan. In some examples, the care plan includes a checklist 218 that is populated and is to be followed daily (or other time period) by a caregiver.

In some examples, a control interface 216 is configured to allow interaction with the LMT system 200. For example, the control interface 216 includes one or more user interfaces configured to receive the input data 204 (e.g., LMT domain expert data), the assessment responses 212, as well as other responses, such as feedback as described in more detail herein. That is, the control interface 216 allows for user inputs or other inputs to be received and utilized by the LMT assessment template processor 202 in assessment template generation and/or assessment execution based on the generated assessment template (e.g., the LMT assessment template 208) and corresponding care plan (e.g., one or more care plan checklist(s) 218).

Reponses to one or more items in the checklist 218 (e.g., activities to be performed, tasks to be performed, etc.) in some examples are received manually by a user input or automatically from one or more monitoring devices. That is, measured or observed activities 220 are provided as feedback 222 to the LMT assessment template processor 202. For example, with the input data 202 processed as described above, the LMT assessment template processor 202 generates the LMT assessment template 208, which is used to produce the output 210 by the rules engine 214 and based on the assessment responses 212. The feedback 222 is then used in various examples to confirm performance of the items, which in some examples, includes confirming that one or more items on the checklist 218 and/or that further actions/tasks are to be performed.

Thus, with the checklist(s) 218 generated as the output 218, LMT monitoring can be performed with feedback provided by one or more devices, such as end user devices, for example, a smart phone 224, a laptop computer 226, or other end user computing device, which allow for user input feedback. In some examples, one or more monitoring devices, such as a camera 228 (or other imaging or non-imaging sensor) and/or a robot 230 are configured to acquire information for the measured and/or observed activities corresponding to one or more items on the checklist(s) 218 and automatically provide this information as the feedback 222. It should be noted that in some examples, the LMT assessment template generator 100 or components thereof are configured as a downloadable application that can be stored and loaded to one or more of the end user devices. The end user device is able to use the LMT assessment template generator 100 to generate one or more assessment templates, corresponding checklist(s) 218, and/or provide feedback 222.

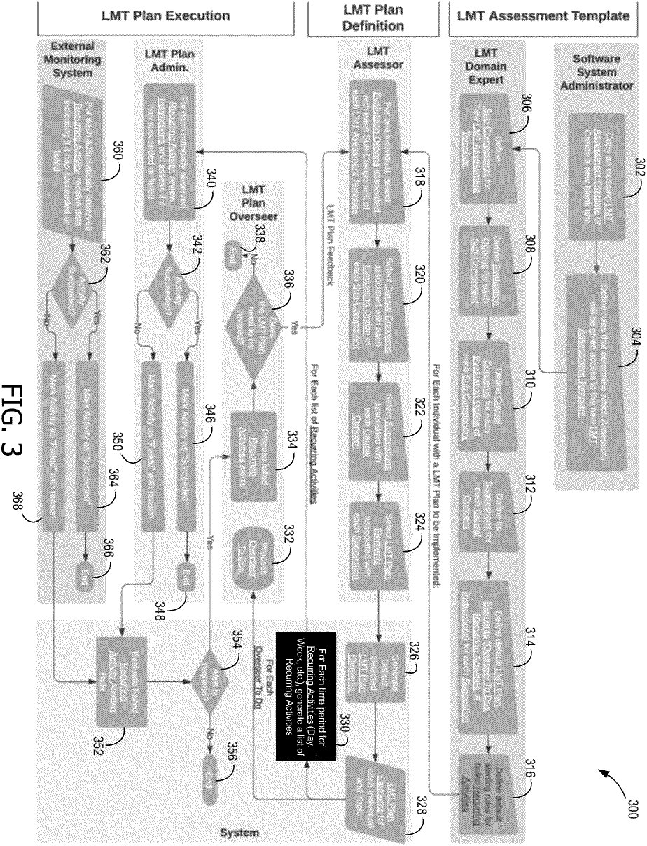

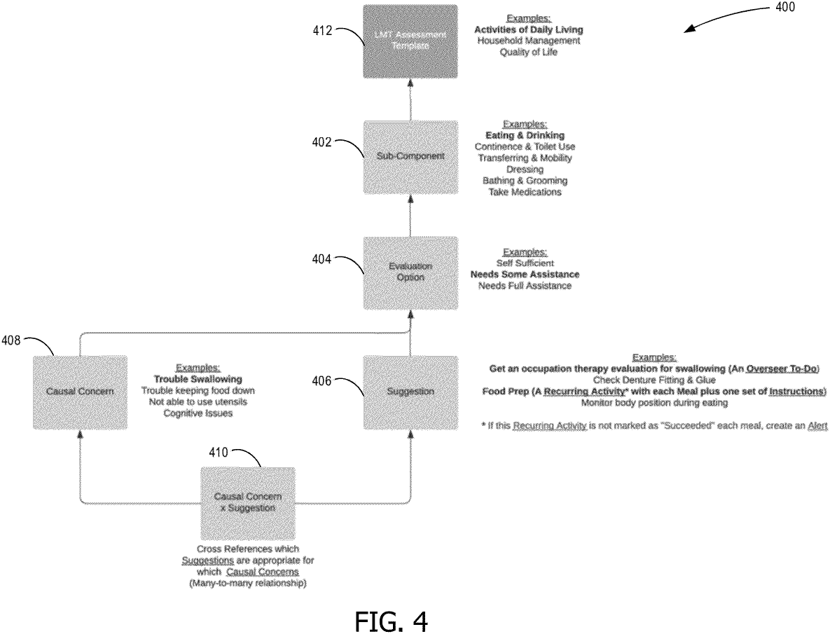

In some implementations, the LMT assessment template generator 100 and/or the LMT system 200 is operable and configured to perform one or more of an LMT assessment template and care plan generation and execution process as illustrated in FIG. 3 using an LMT assessment template data structure 400 as illustrated in FIG. 4. In particular, a flowchart 300 of a process flow illustrating operations involved in generation of an LMT assessment template (operations 302-316), defining an LMT plan (e.g., LMT care plan) based on the LMT assessment template (operations 318-330), and execution of the LMT plan (operations 332-368) is shown in FIG. 3. With the process illustrated by the flowchart 300, in various examples, templated life management assessments and recurring activities management can be performed, which includes dynamic care plans.

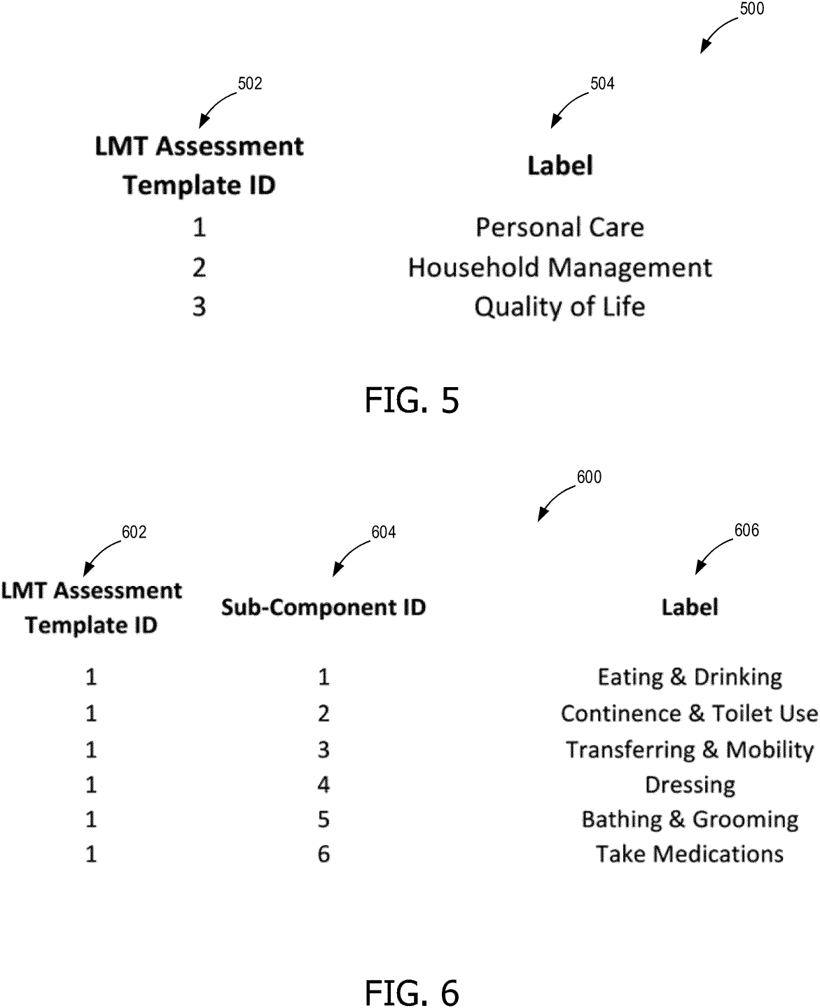

The flowchart 300 commences with operation 302 that includes initiating template generation at 302 by selecting an existing LMT assessment template (e.g., a previously generated template) or creating a new blank template. If an existing LMT assessment template is selected (e.g., copied from a template database), a template structure is already defined and the process 300 allows for modification of the template structure, such as to be customized to a present environment or need. At operation 304, rules are defined that determine which assessors will be given access to the new LMT assessment template. For example, access rights to the LMT assessment template can be set. In various examples, operations 302 and 304 involve receiving inputs from a software system administrator. That is, the selection of a blank or existing template and the setting of access rights is based on inputs from the software system administrator. In one example, a template definition interface 500 as shown in FIG. 5 allows for defining and/or selecting a defined (or existing) LMT assessment template, which in this example includes three different templates identified by an LMT assessment template identification (ID) 502 (e.g., template number) and a label 504 for the LMT assessment template (e.g., corresponding name or description for the LMT assessment template ID). It should be noted that while the illustrated LMT assessment templates are related to personal care, household management, and quality of life, other templates can be provided and/or selected. Additionally, in some examples, a blank template selection is also provided.

With the template selected, the elements of the template are defined or modified, which involves receiving inputs (at operations 306-316) from an LMT domain expert in some examples. More particularly, at 306 one or more sub-components for the LMT assessment template are defined. For example, as illustrated in FIG. 6, a sub-component definition interface 600 allows for defining one or more sub-components within a defined LMT assessment template. In this example, the sub-components are associated with one of the LMT assessment templates as identified by the LMT assessment template identification (ID) 602 and identified by a sub-component ID 604 (e.g., a sub-component reference number) and a label for the sub-components 606 (e.g., description of the defined sub-component). That is, the label defines each of a plurality of sub-components 606, which in this example relate to a personal care LMT assessment template. In this example, the sub-components 606 define categories of activities relating to personal care. In some examples, the sub-components 606 are defined by the LMT domain expert, which can be an expert in the field, an experienced individual in the field, etc. In some examples, the sub-components 606 are defined by a computer algorithm, AI, or other type of machine learning that generates the sub-components 606. For example, one or more suggested sub-components 606 can be generated by an automated process, such as based on past sub-components 606, known tasks in the field, known issues in the field, etc.

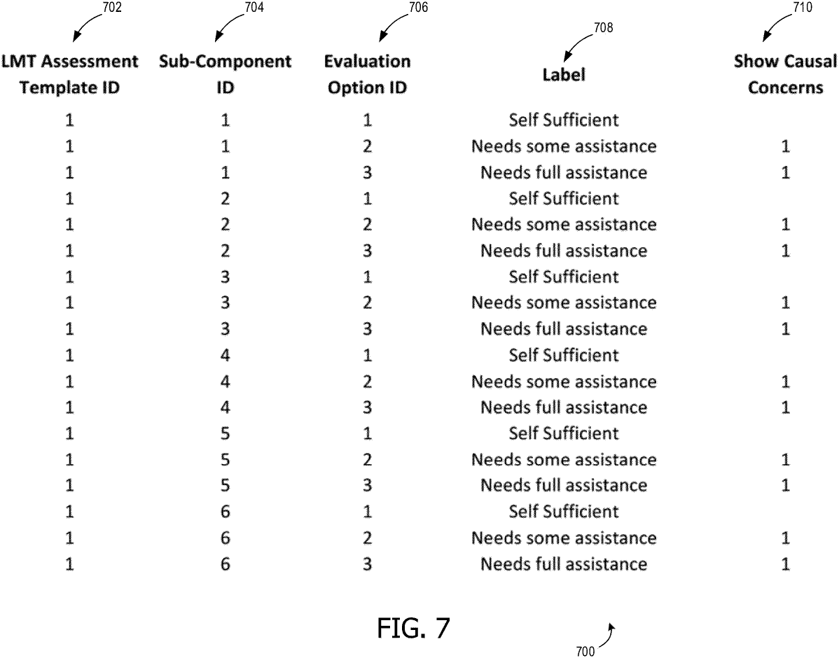

At 308, one or more evaluation options are defined for each sub-component 606. For example, as illustrated in FIG. 7, an evaluation option definition interface 700 allows for defining one or more evaluation options for each sub-component 606 within a defined LMT assessment template. In this example, the one or more evaluation options are associated with one of the LMT assessment templates as identified by the LMT assessment template identification (ID) 702 and the one of the sub-components 606 as identified by the sub-component ID 704. Each of the evaluation options is identified by an evaluation option ID 706 and a label for the evaluation options 708. That is, the label defines each of a plurality of evaluation options 708, which in this example relate to the personal care LMT assessment template. In this example, the evaluation options 708 define levels of sufficiency for each of the categories of activities relating to personal care. For example, different levels of sufficiency for each sub-component 606 can be defined.

It should be noted that although the illustrated example includes three levels of defined sufficiency, fewer or additional levels can be provided, such as based on a desired level of granularity in sufficiency performance. In the illustrated example, levels below full sufficiency or self-sufficiency, or below a defined sufficiency threshold or level can be identified as being in a show causal concern category 710, which results in causal concerns being defined or identified for each of the identified sufficiency levels. That is, for any sufficiency level below a defined threshold, in this example “self-sufficient”, additional definitions are provided as causal concerns, which are performed at 310.

In some examples, the evaluation options 708 are defined by an LMT domain expert, which can be an expert in the field, an experienced individual in the field, etc. In some examples, the evaluation options 708 are defined by a computer algorithm, AI, or other type of machine learning that generates evaluation options 708. For example, one or more suggested evaluation options 708 can be generated by an automated process, such as based on past evaluation options 708, the category defined by the corresponding sub-component 606, known tasks in the field, known issues in the field, etc.

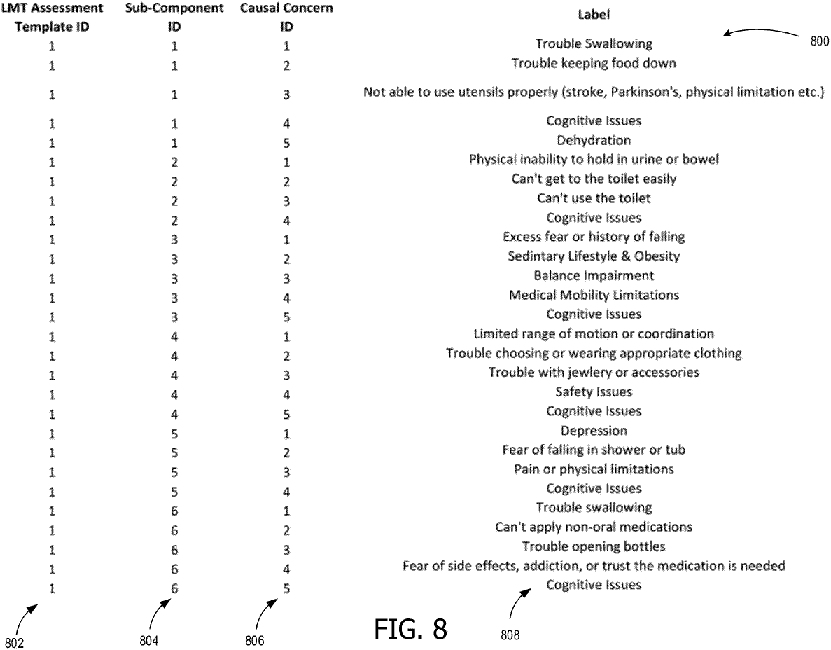

With particular reference now to operation 310, one or more causal concerns are defined for each evaluation option 708 within each sub-component 606, wherein the evaluation option is below a defined threshold. For example, as illustrated in FIG. 8, a causal concern definition interface 800 allows for defining one or more causal concerns for each evaluation option 708 below the defined threshold in the sub-component 606. In this example, the one or more causal concerns are associated with one of the LMT assessment templates as identified by the LMT assessment template identification (ID) 802, the one of the sub-components 606 as identified by the sub-component ID 804, and a corresponding one of the causal concerns below the defined threshold as identified by a causal concern ID 806. That is, each of the evaluation options is identified by the causal concern ID 806 and a label for the causal concerns 808. The label defines each of a plurality of causal concerns 808, which in this example relate to the personal care LMT assessment template. It should be noted that in various examples, the IDs can be numbers associated with the various components, such as the corresponding elements in the hierarchical or tree structure being defined.

In this example, the causal concerns 808 are defined only for levels of sufficiency for each of the categories of activities relating to personal care that are below the defined threshold level. As discussed herein, in the illustrated example, levels below full sufficiency or self-sufficiency, that are identified as being in a show causal concern category 710, results in the causal concerns 808 being defined or identified for each of the identified sufficiency levels for the sub-components 606. That is, for each sub-component 606 having an evaluation option 708 below the defined threshold, additional elements can be defined for each, which in this example are the causal concerns 808. As can be seen, the causal concerns 808 for each of the evaluation options 708 in the different sub-components 606 can be the same or different.

In some examples, the causal concerns 808 are defined by an LMT domain expert, which can be an expert in the field, an experienced individual in the field, etc. In some examples, the causal concerns 808 are defined by a computer algorithm, AI, or other type of machine learning that generates causal concerns 808. For example, one or more suggested causal concerns 808 can be generated by an automated process, such as based on past causal concerns 808, the category defined by the corresponding sub-component 606, the evaluation option 708, known tasks in the field, known issues in the field, etc.

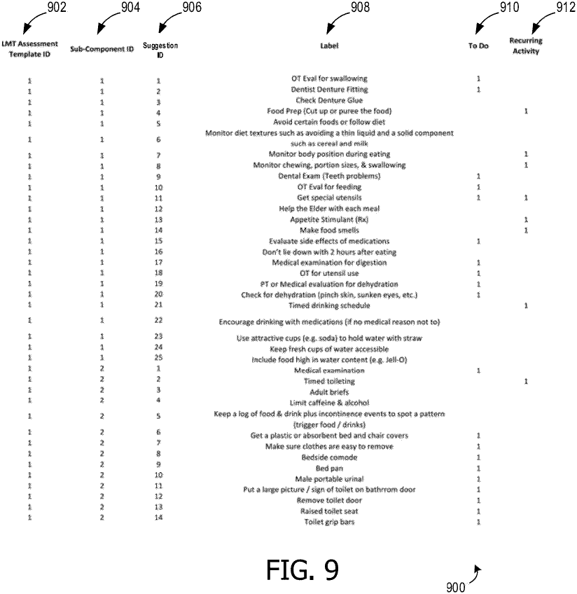

At 312, one or more suggestions are defined for each causal concern 808 within each sub-component 606, wherein the evaluation option is below the defined threshold. For example, as illustrated in FIG. 9 a suggestion definition interface 900 allows for defining the one or more suggestions for each causal concern 808 corresponding to an evaluation option 708 below the defined threshold in the sub-component 606. In this example, the one or more suggestions are associated with one of the LMT assessment templates as identified by an LMT assessment template identification (ID) 902, the one of the sub-components 606 as identified by a sub-component ID 904, and a corresponding suggestion as identified by a suggestion ID 906. That is, each of the suggestions is identified by the suggestion ID 906 and a label for the suggestions 908. The label defines each of a plurality of suggestions 908, which in this example relate to the personal care LMT assessment template.

In this example, the suggestions 908 are defined only for causal concerns 808 that are for levels of sufficiency for each of the categories of activities relating to personal care that are below the defined threshold level. That is, for each sub-component 606 having an evaluation option 708 below the defined threshold, wherein additional elements are defined for each, namely the causal concerns 808, the one or more suggestions 908 are further defined. The one or more suggestions 908 for each of the causal concerns 808 in the different sub-components 606 can be the same or different.

In some examples, the suggestions 908 are defined by an LMT domain expert, which can be an expert in the field, an experienced individual in the field, etc. In some examples, the suggestions 908 are defined by a computer algorithm, AI, or other type of machine learning that generates causal concerns 808. For example, the one or more suggestions 908 can be generated by an automated process, such as based on past suggestions 908, the causal concerns 808, the category defined by the corresponding sub-component 606, the evaluation option 708, known tasks in the field, known issues in the field, etc. It should be noted that the suggestions illustrated in FIG. 9 are merely for example and any suggestion relating to the particular application can be defined. That is, a preset list of suggestions, while generated in some examples (e.g., by machine learning), does not limit adding suggestions or modifying the suggestions 908.

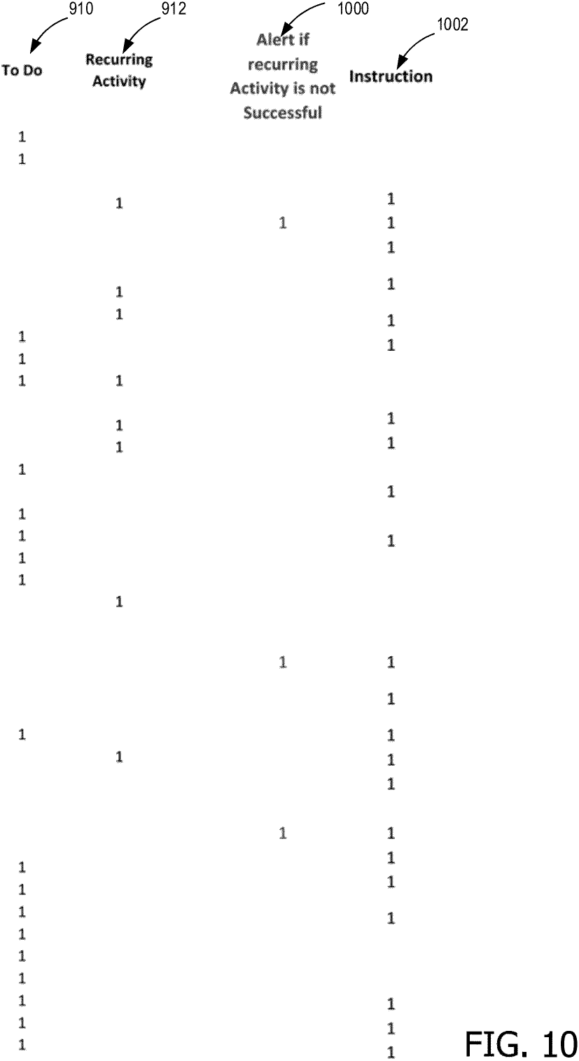

In this example, at 314, default LMT plan elements (e.g., care plan elements) for each of the suggestions 908 are defined. For example, overseer to do action items (e.g., “To Do” action 910, “Recurring Activity” action 912, alert action 1000 and/or instruction action 1002 as illustrated in FIGS. 9 and 10) and recurring activity action items (and instructions) are defined for each of the suggestions. In one example, the suggestion definition interface 900 in the illustrated example allows for defining or categorizing each of the suggestions 908 as an item (e.g., activity or task) that needs to be done once for the causal concerns 808 indicated as a “To Do” action 910 or an item that needs to be done multiple times for the causal concerns 808, such as on a recurring basis, indicated as a “Recurring Activity” action 912. As such, different levels or frequency of the action corresponding to each of the suggestions 908 can be defined. For example, one or more actions, tasks, activities, etc. to be performed with respect to the suggestions 908 can be identified using the “To Do” action 910 or the “Recurring Activity” action 912.

In some examples, alerting rules are defined at 316. For example, default alerting rules for failed recurring activities are defined. The suggestion definition interface 900 is configured to receive inputs corresponding to the alerting rules in one example. As can be seen in FIG. 10, in addition to the “To Do” action 910 and the “Recurring Activity” action 912, an alert action 1000 and an instruction action 1002 can further be defined. For example, the alert action 1000 and an instruction action 1002 define alerts and instructions, respectively, for the suggestions 908. In some examples, the alert action 1000 defines default alerting rules, such that any of the suggestions 908 defined to have an alert condition based on the alert action 1000 (illustrated by a “1” in this example), results in an alert being generated if the activity is not successfully performed, such as if the suggestion 908 is not properly performed, not satisfactorily performed, not performed within a defined time period, etc. As described in more detail herein, the alert can be generated in response to received feedback, such as from a caregiver, sensor, etc.

At this point, after completing operation 316, the LMT assessment template (e.g., the LMT assessment template 208) is defined. That is, the elements for the particular LMT assessment template, in this example, for elder care, is defined. As such, the components, action items, etc. for the LMT assessment template are now defined and can be used, for example, to generate the LMT plan, such as the acer plan. For example, using the LMT assessment template, the LMT plan can be defined, such as generated from the LMT assessment template. As one example, a customized care plan (e.g., elder care plan) is generated from the LMT assessment template, which includes for an individual, selecting one or more evaluation options 708 associated with each sub-component 606 for the LMT assessment template being used. That is, user inputs (e.g., healthcare provider) are received at 318 to select the evaluation options 708, such as selecting from the defined evaluation options 708 (e.g., selecting from drop-down menus of a user interface (such as the control interface 216), selecting items in a checklist, entering inputs into selection fields, etc.). The selection of the evaluation options 708 results in the LMT plan including the selected evaluation options 708.

Causal concerns 808 associated with each of the evaluation options 708 for each sub-component 606 are then selected at 320. For example, user inputs are received at a user interface selecting one or more defined causal concerns 808 for the customized care plan that will be generated from the LMT assessment template. For example, the causal concerns 808 are selected in some examples based on the person for whom care is to be provided, the requirements of the care facility, etc. It should be appreciated that the selections, for example, user inputs received at the user interface, can be changed, such as based on changing care needs, etc.

Suggestions 908 associated with the causal concerns 808 for each of the evaluation options 708 for each sub-component 606 are then selected at 322. For example, user inputs are received at a user interface selecting one or more defined suggestions for the customized care plan that will be generated from the LMT assessment template. For example, the suggestions 908 are selected in some examples based on the person for whom care is to be provided, the requirements of the care facility, etc. It should be appreciated that the selections, for example, user inputs received at the user interface, can be changed, such as based on changing care needs, etc.

LMT plan elements for each of the suggestions 908 associated with the causal concerns 808 for each of the evaluation options 708 for each sub-component 606 are then selected at 324. For example, user inputs are received selecting one or more action items (e.g., “To Do” action 910, “Recurring Activity” action 912, alert action 1000 and/or instruction action 1002) for the customized LMT plan that will be generated from the LMT assessment template. For example, the one or more action items are selected in some examples based on the person for whom care is to be provided, the requirements of the care facility, etc. It should be appreciated that the selections, for example, user inputs received at the user interface, can be changed, such as based on changing care needs, etc.

Default selected LMT plan elements are then generated at 326. For example, elements to include in an elder care plan based on the received selections at operations 318, 320, 322, and 324 are configured such that LMT plan elements for the individual (or multiple individuals) and topic are output at 328. For example, one or more customized checklists or action item lists can then be generated. In one example, at 330, for each time period for recurring activities that are defined in the LMT assessment template, a list of recurring activities (include day, week, etc.) is generated. It should be appreciated that the various outputs, plans, checklists, etc. can be generated and provided electronically (e.g., displayed on a user interface or device) or in physical form (e.g., a printed checklist).

Additionally, in response to the operation at 328, overseer to do actions (e.g., “To Do” actions 910 defined by the LMT assessment template and selected for the LMT plan) are processed at 332. That is, action items specifically designated for the overseer versus the caregiver are processed to generate, for example, an action item list.

With the LMT plan defined and corresponding outputs generated, such as action or activities lists, task lists, etc., feedback is received (as part of the LMT plan execution). The feedback in various examples is received manually, such as via user input (e.g., LMT plan administrator or caregiver input via a user interface), or automatically, such as via one or more monitoring devices (e.g., movement monitor, physiological monitor, etc.), sensors, etc. For each manually entered feedback, such as manually observed recurring activity, at 340, the instructions (e.g., the instruction 1002) are reviewed and assessed to determine whether the activity has succeeded or failed. For example, a determination is made at 342 whether the defined threshold level as described herein has been met (e.g., feedback indicates that the evaluation response is self-sufficient and not a “needs some assistance” or “needs full assistance” response). If the activity succeeded, then the activity is marked as “succeeded” at 346 and the process with respect to that recurring activity ends at 348. If the activity did not succeed, then the activity is marked as “failed” with a reason for failure indicated at 350.

Similarly, for each automatically observed recurring activity, data is received at 360 indicating if the recurring activity has succeeded or failed. For example, observed monitoring data (e.g., physical monitoring data, image data, etc.) indicative of success or failure of the activity is received. For example, a determination is made at 362 whether the defined threshold level as described herein has been met (e.g., feedback indicates that the evaluation response is self-sufficient and not a “needs some assistance” or “needs full assistance response”). If the activity succeeded, then the activity is marked as “succeeded” at 364 and the process with respect to that recurring activity ends at 366. If the activity did not succeed, then the activity is marked as “failed” with a reason for failure indicated at 368.

In response to the activity being marked as “failed” at 350 or 368, the failed recurring activity/alerting rule is evaluated at 352. For example, a determination is made whether the alert is required at 354. In one example, this determination includes whether the recurring activity is marked in the assessment template as requiring alerting at 316. If not marked, the alert is not required, and the process ends at 356. If the alert is still required, then at 334 the failed recurring activity alert is processed at 334 to determine whether the LMT plan needs to be revised or modified at 336. For example, a determination is made as to whether there is a change in health status of the individual who is the subject of the LMT plan, a change in facility, a change in available actions, etc. If a determination is made that no change in the LMT plan is needed, then the process ends at 338. If a determination is made at 336 that a change in the LMT plan is needed, then the process returns to operation 318 to again select evaluation options.

In various examples, the process performed by the flowchart 300 and the user interfaces used, outputs generated, etc. use the LMT assessment template data structure 400 illustrated in FIG. 4. That is, the LMT assessment template data structure 400 includes data (e.g., LMT domain expert input data) configured for accessing and processing by the flowchart 300 to generate the LMT assessment template and corresponding LMT plans. The LMT assessment template data structure 400 defines a hierarchical data structure that allows for providing the various interfaces that receive the inputs for generating the LMT assessment template. In the illustrated example, a top level of an LMT assessment template 412 is sub-component data 402. In various examples, the sub-component data 402 corresponds to the sub-components 606. The next level below the sub-component data 402 is evaluation option data 404 that corresponds to the evaluation options 708. The next level below the evaluation option data 404 is the suggestion data 406 that corresponds to the suggestions 908 and causal concern data 408 that corresponds to the causal concerns 808.

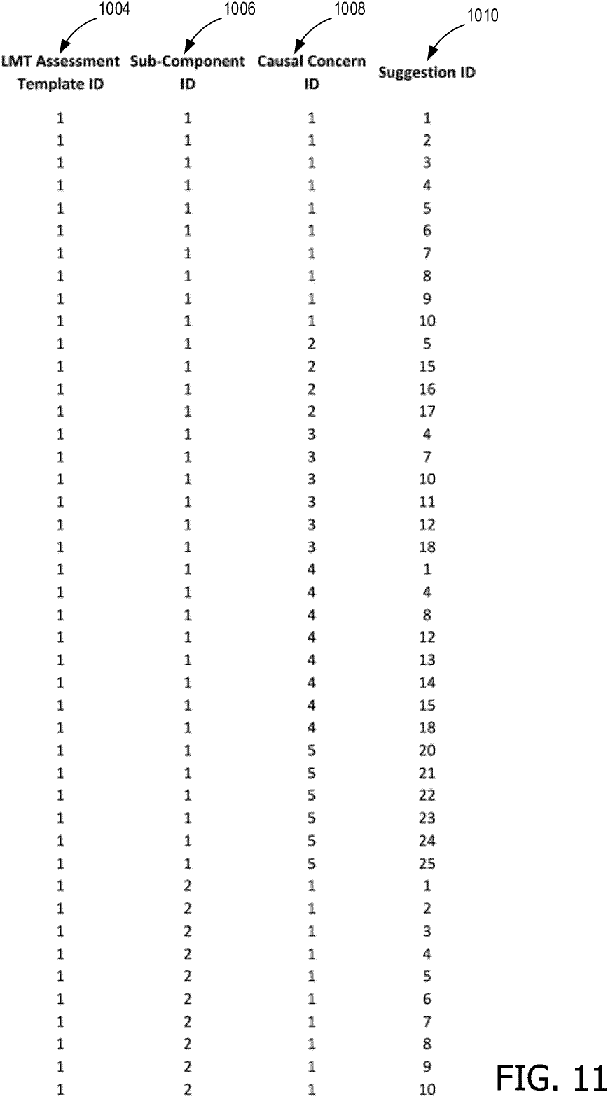

The next level below the suggestion data 406 and the causal concern data 408 is cross-reference data 410. That is, the cross-reference data 410 is related to the suggestion data 406 and the causal concern data 408. In one example, the cross-reference data 410 cross-references which suggestions 908 are appropriate for which causal concerns 808 in a many-to-many relationship. For example, based on the type of data or content data, or other metadata related to the suggestions 908 and causal concerns 808, cross-reference of related topics, content, etc. are determined and the corresponding relationships stored. FIG. 11 illustrates correlated data identifiers for the example described herein. As can be seen, an LMT assessment template ID 1004, a sub-component ID 1006, a causal concern ID 1008, and a suggestion ID 1010 are correlated (e.g., each row represents a correlation with the ID numbers corresponding to the example described herein). It should be noted that the IDs 1004, 1006, 1008, and 1010 correspond to the IDs 502, 604, 806, and 906, respectively.

Thus, one or more examples utilize a data driven implantation to define the input data flow that results in the output. In one example, the input allows a user to select from options (such as multiple options) and yes/no questions. The process flow is dynamic in that the process flow is not hard-wired. For example, answers may drive new questions, and templates are customizable. For instance, on the input side, the user interface can be configured to take into consideration the training level of the “overseer” or person answering the questions, such that lay people view ordinary speech/language and professionals view terms of art, so that the vernacular or context is changed. Thus, the process flow is not static, thereby resulting in improved template generation for many different environments.

As described herein, the output is customizable, such as based on the elder and the care giving space (e.g., home, assisted living, etc.). The customized output is connected to the list of activities to create checklist entries, which for example, can repeat daily with needed variations (e.g., physical therapy (PT) on Wednesdays). Thus, a dynamic and customizable process that includes a workflow of template input → template output → checklists (e.g., take medications, eat breakfast, go on walk, occupational therapy (OT), lunch, etc.) is provided. As described herein, alerts can be provided, for example, to the “overseer” (e.g., text or email) if an item on the checklist is not performed or not satisfactorily performed. As a result, a person’s care or a house may be managed, medications taken, appointments kept, etc. That is, checklists are just in time activity lists with dynamic real-timeliness that allow for care planning and flow control.

With the LMT assessment templates that are used to generate care plans, one or more “care plan routines” can be developed. While the examples are described in connection with care plans, one or more examples can be implemented in a management setting with coordination among all involved parties.

Additionally, in various examples, as described herein, updates and revisions can be easily made. For example, medications are typically taken in groups at certain times (e.g., with meals, upon waking or going to bed, etc.). Medications and instructions are entered as part of assessment feedback in some examples. The system then groups the feedback into time events and automatically lists the events with appropriate instructions in checklists. If there is a change in one or more medications, a user can make an edit in the system and the system automatically adjusts the recurring activities to reflect the change. In some examples, an answer to the original assessment can be manually changed and recurring validation with periodic reassessments also can be performed (e.g., every three months). In some examples, one-time tasks or actions can be added, such as entering items (e.g., to make an appointment to have hearing checked).

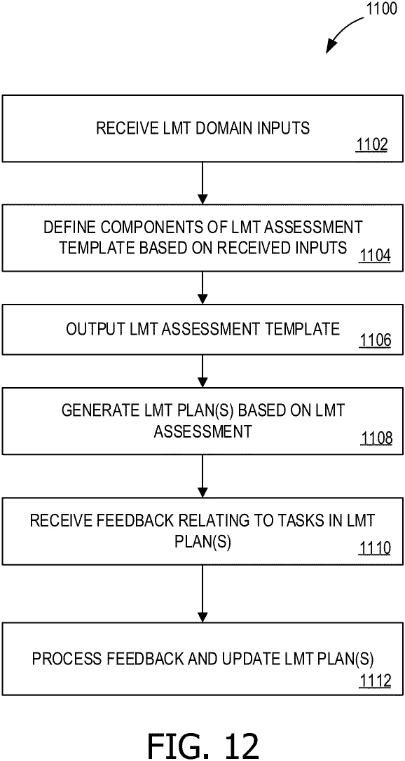

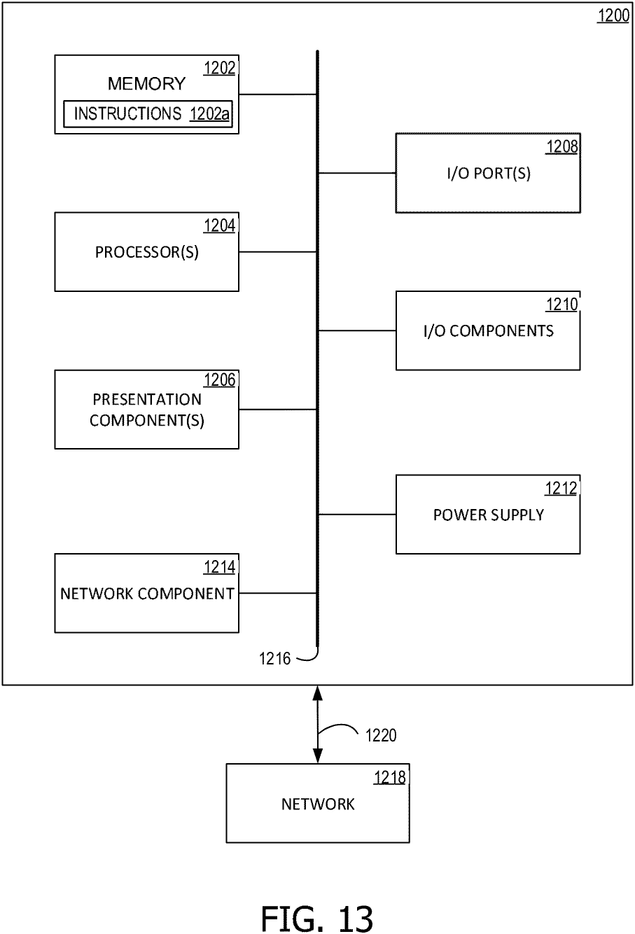

FIG. 12 is a flowchart 1100 illustrating operations involved in LMT assessment template and LMT plan definition and updating. In some examples, the operations of the flowchart 1100 are performed by the LMT assessment template generator 100, the LMT system 200, and/or the computing device 1200 illustrated in FIG. 13 (which may form part of or implement part of the LMT assessment template generator 100 and/or the LMT system 200). The flowchart 1100 commences with operation 1102 that includes receiving a plurality of inputs, which in various examples are a plurality of LMT domain expert inputs. For example, inputs relating to elder care are received from one or more LMT experts. The inputs are received via a user interface in some examples, wherein the user interface can have predefined categories, such as predefined sub-components 606. In other examples, predefined categories or other guidance can be provided. In some examples, a blank template input interface is provided with a template name indicating the type of LMT plan(s) to be generated from the template (e.g., personal care, household management, quality of life, etc.). It should be noted that the input interface can be any suitable interface that allows user interaction. While a spreadsheet type of input can be used in some examples, other data input techniques and methodologies are likewise suitable and used. In various examples, query operations are performed as part of the data collection instead of manual entry in a spreadsheet expert system.

The LMT domain inputs are stored, for example, as an expert data collection in a database (e.g., a database structure that drives a user interface for collecting information) and used to define components of the LMT assessment template at 1104. For example, the data inputs are used in various examples to generate the various components of the input interfaces described herein (e.g., interfaces 500, 600, 700, 800, and 900). In some examples, the data input is used to generate metadata that creates the assessment template. As should be appreciated, the input methodology and generation of templates is thereby data driven and not hard-coded in software. That is, a dynamic assessment template and plan generation methodology as described herein allows for customizable assessments in any domain, such as for different life management assessments. The example of the LMT domain is merely for illustration of one or more inventive aspects.

An LMT assessment template is then output at 1106. For example, one or more templates generated based on the received inputs are output for use in generating one or more LMT plans (e.g., elder care or health care plans) at 1108. In some examples, selections available within the various interfaces are based on the defined components and allows for receiving user inputs in the LMT assessment template. As described herein, the LMT assessment template in various examples is a relational template that is driven by a hierarchical data structure (e.g., the LMT assessment template data structure 400) and allows for generating customized LMT plans based on the needs of the individual, requirements for care, etc. In one example, the generation of the customized LMT plan(s) includes one or more data driven checklists for elder care. It should be noted that in various examples, the LMT assessment template is defined once with instances of the template thereafter run as needed or desired. In some examples, generic software is used to run the different templates.

Feedback relating to tasks with the LMT plans is received at 1110. For example, task performance data is received from one or more individuals (e.g., caregivers) or from one or more devices (e.g., sensors or a robot). That is, the feedback defines user data collected that can also be stored in the database. As described herein, the feedback in some examples relates to whether one or more tasks of the LMT plan(s) have been performed at a defined level (e.g., self-sufficient). It should be noted that the feedback can relate to tasks performed by any individual, including, for example, performance by the caregiver, performance by the individual receiving care, etc.

The feedback is processed at 1112 and any updates to the LMT plan(s) are made. For example, the assessments to be performed, the frequency of the assessments, etc. can be changed, added, and/or removed based on the received feedback. In some examples, the updates are performed automatically based on the received feedback (which in one example requires user confirmation). In other examples, the updates are performed manually by a user based on the received feedback.

Thus, various examples provide assessment template and plan definition and execution, such as within the LMT domain. As described herein, one or more examples provide data driven templated LMT assessments and recurring activities management using one or more data driven systems.

With reference now to FIG. 13, a block diagram of the computing device 1200 suitable for implementing various aspects of the disclosure is described (e.g., the LMT system 200). FIG. 13 and the following discussion provide a brief, general description of a computing environment in/on which one or more or the implementations of one or more of the methods and/or system set forth herein may be implemented. The operating environment of FIG. 13 is merely an example of a suitable operating environment and is not intended to suggest any limitation as to the scope of use or functionality of the operating environment. Example computing devices include, but are not limited to, personal computers, server computers, hand-held or laptop devices, mobile devices (such as mobile phones, mobile consoles, tablets, media players, and the like), multiprocessor systems, consumer electronics, mini computers, mainframe computers, distributed computing environments that include any of the above systems or devices, and the like.

Although not required, implementations are described in the general context of “computer readable instructions” executed by one or more computing devices. Computer readable instructions may be distributed via computer readable media (discussed below). Computer readable instructions may be implemented as program modules, such as functions, objects, Application Programming Interfaces (APIs), data structures, and the like, that perform particular tasks or implement particular abstract data types. Typically, the functionality of the computer readable instructions may be combined or distributed as desired in various environments.

In some examples, the computing device 1200 includes a memory 1202, one or more processors 1204, and one or more presentation components 1206. The disclosed examples associated with the computing device 400 are practiced by a variety of computing devices, including personal computers, laptops, smart phones, mobile tablets, hand-held devices, consumer electronics, specialty computing devices, etc. Distinction is not made between such categories as “workstation,” “server,” “laptop,” “hand-held device,” etc., as all are contemplated within the scope of FIG. 13 and the references herein to a “computing device.” The disclosed examples are also practiced in distributed computing environments, where tasks are performed by remote-processing devices that are linked through a communications network. Further, while the computing device 1200 is depicted as a single device, in one example, multiple computing devices work together and share the depicted device resources. For instance, in one example, the memory 1202 is distributed across multiple devices, the processor(s) 1204 provided are housed on different devices, and so on.

In one example, the memory 1202 includes any of the computer-readable media discussed herein. In one example, the memory 1202 is used to store and access instructions 1202a configured to carry out the various operations disclosed herein. In some examples, the memory 1202 includes computer storage media in the form of volatile and/or nonvolatile memory, removable or non-removable memory, data disks in virtual environments, or a combination thereof. In one example, the processor(s) 1204 includes any quantity of processing units that read data from various entities, such as the memory 1202 or input/output (I/O) components 1210. Specifically, the processor(s) 1204 are programmed to execute computer-executable instructions for implementing aspects of the disclosure. In one example, the instructions 1202a are performed by the processor 1204, by multiple processors within the computing device 1200, or by a processor external to the computing device 1200. In some examples, the processor(s) 1204 are programmed to execute instructions such as those illustrated in the flow charts discussed herein and depicted in the accompanying drawings.

In other implementations, the computing device 1200 may include additional features and/or functionality. For example, the computing device 1200 may also include additional storage (e.g., removable and/or non-removable) including, but not limited to, magnetic storage, optical storage, and the like. Such additional storage is illustrated in FIG. 13 by the memory 1202. In one implementation, computer readable instructions to implement one or more implementations provided herein may be in the memory 1202 as described herein. The memory 1202 may also store other computer readable instructions to implement an operating system, an application program and the like. Computer readable instructions may be loaded in the memory 1202 for execution by the processor(s) 1204, for example.

The presentation component(s) 1206 present data indications to an operator or to another device. In one example, the presentation components 1206 include a display device, speaker, printing component, vibrating component, etc. One skilled in the art will understand and appreciate that computer data is presented in a number of ways, such as visually in a graphical user interface (GUI), audibly through speakers, wirelessly between the computing device 1200, across a wired connection, or in other ways. In one example, the presentation component(s) 1206 are not used when processes and operations are sufficiently automated that a need for human interaction is lessened or not needed. I/O ports 1208 allow the computing device 1200 to be logically coupled to other devices including the I/O components 1210, some of which is built in. Implementations of the I/O components 1210 include, for example but without limitation, a microphone, keyboard, mouse, joystick, pen, game pad, satellite dish, scanner, printer, wireless device, camera, etc.

The computing device 1200 includes a bus 1216 that directly or indirectly couples the following devices: the memory 1202, the one or more processors 1204, the one or more presentation components 1206, the input/output (I/O) ports 1208, the I/O components 1210, a power supply 1212, and a network component 1214. The computing device 1200 should not be interpreted as having any dependency or requirement related to any single component or combination of components illustrated therein. The bus 1216 represents one or more busses (such as an address bus, data bus, or a combination thereof). Although the various blocks of FIG. 13 are shown with lines for the sake of clarity, some implementations blur functionality over various different components described herein.

The components of the computing device 1200 may be connected by various interconnects. Such interconnects may include a Peripheral Component Interconnect (PCI), such as PCI Express, a Universal Serial Bus (USB), firewire (IEEE 1394), an optical bus structure, and the like. In another implementation, components of the computing device 1200 may be interconnected by a network. For example, the memory 1202 may be comprised of multiple physical memory units located in different physical locations interconnected by a network.

In some examples, the computing device 1200 is communicatively coupled to a network 418 using the network component 1214. In some examples, the network component 1214 includes a network interface card and/or computer-executable instructions (e.g., a driver) for operating the network interface card. In one example, communication between the computing device 1200 and other devices occurs using any protocol or mechanism over a wired or wireless connection 1220. In some examples, the network component 1214 is operable to communicate data over public, private, or hybrid (public and private) connections using a transfer protocol, between devices wirelessly using short range communication technologies (e.g., near-field communication (NFC), Bluetooth® branded communications, or the like), or a combination thereof.

The connection 1220 may include, but is not limited to, a modem, a Network Interface Card (NIC), an integrated network interface, a radio frequency transmitter/receiver, an infrared port, a USB connection or other interfaces for connecting the computing device 1200 to other computing devices. The connection 1220 may transmit and/or receive communication media.

Although described in connection with the computing device 1200, examples of the disclosure are capable of implementation with numerous other general-purpose or special-purpose computing system environments, configurations, or devices. Implementations of well-known computing systems, environments, and/or configurations that are suitable for use with aspects of the disclosure include, but are not limited to, smart phones, mobile tablets, mobile computing devices, personal computers, server computers, hand-held or laptop devices, multiprocessor systems, gaming consoles, microprocessor-based systems, set top boxes, programmable consumer electronics, mobile telephones, mobile computing and/or communication devices in wearable or accessory form factors (e.g., watches, glasses, headsets, or earphones), network PCs, minicomputers, mainframe computers, distributed computing environments that include any of the above systems or devices, VR devices, holographic device, and the like. Such systems or devices accept input from the user in any way, including from input devices such as a keyboard or pointing device, via gesture input, proximity input (such as by hovering), and/or via voice input.

Implementations of the disclosure are described in the general context of computer-executable instructions, such as program modules, executed by one or more computers or other devices in software, firmware, hardware, or a combination thereof. In one example, the computer-executable instructions are organized into one or more computer-executable components or modules. Generally, program modules include, but are not limited to, routines, programs, objects, components, and data structures that perform particular tasks or implement particular abstract data types. In one example, aspects of the disclosure are implemented with any number and organization of such components or modules. For example, aspects of the disclosure are not limited to the specific computer-executable instructions or the specific components or modules illustrated in the figures and described herein. Other examples of the disclosure include different computer-executable instructions or components having more or less functionality than illustrated and described herein. In implementations involving a general-purpose computer, aspects of the disclosure transform the general-purpose computer into a special-purpose computing device when configured to execute the instructions described herein.

By way of example and not limitation, computer readable media comprises computer storage media and communication media. Computer storage media include volatile and nonvolatile, removable, and non-removable memory implemented in any method or technology for storage of information such as computer readable instructions, data structures, program modules, or the like. Computer storage media are tangible and mutually exclusive to communication media. Computer storage media are implemented in hardware and exclude carrier waves and propagated signals. Computer storage media for purposes of this disclosure are not signals per se. In one example, computer storage media include hard disks, flash drives, solid-state memory, phase change random-access memory (PRAM), static random-access memory (SRAM), dynamic random-access memory (DRAM), other types of random-access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transmission medium used to store information for access by a computing device. In contrast, communication media typically embody computer readable instructions, data structures, program modules, or the like in a modulated data signal such as a carrier wave or other transport mechanism and include any information delivery media.

While various spatial and directional terms, including but not limited to top, bottom, lower, mid, lateral, horizontal, vertical, front and the like are used to describe the present disclosure, it is understood that such terms are merely used with respect to the orientations shown in the drawings. The orientations can be inverted, rotated, or otherwise changed, such that an upper portion is a lower portion, and vice versa, horizontal becomes vertical, and the like.

The word “exemplary” is used herein to mean serving as an example, instance or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims. Of course, those skilled in the art will recognize many modifications may be made to this configuration without departing from the scope or spirit of the claimed subject matter.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein.

Various operations of implementations are provided herein. In one implementation, one or more of the operations described may constitute computer readable instructions stored on one or more computer readable media, which if executed by a computing device, will cause the computing device to perform the operations described. The order in which some or all of the operations are described should not be construed as to imply that these operations are necessarily order dependent. Alternative ordering will be appreciated by one skilled in the art having the benefit of this description. Further, it will be understood that not all operations are necessarily present in each implementation provided herein.

Any range or value given herein can be extended or altered without losing the effect sought, as will be apparent to the skilled person.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure.

As used in this application, the terms “component,” “module,” “system,” “interface,” and the like are generally intended to refer to a computer-related entity, either hardware, a combination of hardware and software, software, or software in execution. For example, a component may be, but is not limited to being, a process running on a processor, a processor, an object, an executable, a thread of execution, a program and/or a computer. By way of illustration, both an application running on a controller and the controller can be a component. One or more components may reside within a process and/or thread of execution and a component may be localized on one computer and/or distributed between two or more computers.

Furthermore, the claimed subject matter may be implemented as a method, apparatus or article of manufacture using standard programming and/or engineering techniques to produce software, firmware, hardware or any combination thereof to control a computer to implement the disclosed subject matter. The term “article of manufacture” as used herein is intended to encompass a computer program accessible from any computer-readable device, carrier or media. Of course, those skilled in the art will recognize many modifications may be made to this configuration without departing from the scope or spirit of the claimed subject matter.

In addition, while a particular feature of the disclosure may have been disclosed with respect to only one of several implementations, such feature may be combined with one or more other features of the other implementations as may be desired and advantageous for any given or particular application. Furthermore, to the extent that the terms “includes,” “having,” “has,” “with,” or variants thereof are used in either the detailed description or the claims, such terms are intended to be inclusive in a manner similar to the term “comprising.”

The implementations have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

What is claimed is:

1. A life management topic (LMT) system, comprising:
a processor; and
a computer-readable medium storing non-transitory instructions that are operative upon execution by the processor to:
receive LMT domain inputs via a user interface;
define components of an LMT assessment template using the received LMT domain inputs;
output the LMT assessment template based at least on the defined components, wherein the LMT assessment template is a relational template having cross-referencing between one or more of the defined components to define a customizable assessment; and
generate one or more LMT plans using the LMT assessment template and based on the customizable assessment.

2. The LMT system of claim 1, wherein the one or more LMT plans comprise a plurality of tasks, and the non-transitory instructions are further operative upon execution by the processor to receive feedback relating to one or more tasks of the plurality of tasks and process the received feedback to generate an alert.

3. The LMT system of claim 2, wherein the one or more LMT plans comprise one or more recurring activities and the non-transitory instructions are further operative upon execution by the processor to generate the alert in response to feedback indicating a failure corresponding to the one or more recurring activities.

4. The LMT system of claim 2, wherein the feedback is received from a manually observed recurring activity.

5. The LMT system of claim 2, wherein the feedback is received from an automatically observed recurring activity, the feedback comprising observation data from one or more of a sensors, a monitor, or a robot.

6. The LMT system of claim 1, wherein the one or more LMT plans comprise a plurality of tasks, and the non-transitory instructions are further operative upon execution by the processor to receive feedback relating one or more tasks of the plurality of tasks and process the received feedback to update the one or more LMT plans.

7. The LMT system of claim 1, wherein the LMT domain inputs comprise one or more definitions for each of sub-components, evaluation options, causal concerns, suggestions, actions items, recurring activities, and instructions for LMT assessment.

8. The LMT system of claim 1, wherein the non-transitory instructions are further operative upon execution by the processor to generate metadata based on the received LMT domain inputs and use the metadata to generate the LMT assessment template.

9. The LMT system of claim 1, wherein the LMT assessment template comprises an elder care template and the one or more LMT plans comprise one or more caregiver checklists generated based at least in part on one or more user inputs relating to suggestions and action items in the elder care template.

10. A computerized method for generating life management topic (LMT) assessment templates, the computerized method comprising:
receiving LMT domain inputs via a user interface;
defining components of an LMT assessment template using the received LMT domain inputs;
outputting the LMT assessment template based at least on the defined components, wherein the LMT assessment template is a relational template having cross-referencing between one or more of the defined components to define a customizable assessment; and
generating one or more LMT plans using the LMT assessment template and based on the customizable assessment.

11. The computerized method of claim 10, wherein the one or more LMT plans comprise a plurality of tasks, and further comprising receiving feedback relating to one or more tasks of the plurality of tasks and process the received feedback to generate an alert.

12. The computerized method of claim 11, wherein the one or more LMT plans comprise one or more recurring activities and further comprise generating the alert in response to feedback indicating a failure corresponding to the one or more recurring activities.

13. The computerized method of claim 11, wherein the feedback is received from a manually observed recurring activity.

14. The computerized method of claim 11, wherein the feedback is received from an automatically observed recurring activity, the feedback comprising observation data from one or more of a sensor, a monitor, or a robot.

15. The computerized method of claim 10, wherein the one or more LMT plans comprise a plurality of tasks, and further comprising receiving feedback relating one or more tasks of the plurality of tasks and process the received feedback to update the one or more LMT plans.

16. The computerized method of claim 10, wherein the LMT domain inputs comprise one or more definitions for each of sub-components, evaluation options, causal concerns, suggestions, actions items, recurring activities, and instructions for LMT assessment.

17. The computerized method of claim 10, further comprising generating metadata based on the received LMT domain inputs and using the metadata to generate the LMT assessment template.

18. The computerized method of claim 10, wherein the LMT assessment template comprises an elder care template and the one or more LMT plans comprise one or more caregiver checklists generated based at least in part on one or more user inputs relating to suggestions and action items in the elder care template.

19. One or more computer storage media having computer-executable instructions for life management topic (LMT) assessment that, upon execution by a processor, cause the processor to at least:
receive LMT domain inputs via a user interface;
define components of an LMT assessment template using the received LMT domain inputs;
output the LMT assessment template based at least on the defined components, wherein the LMT assessment template is a relational template having cross-referencing between one or more of the defined components to define a customizable assessment; and
generate one or more LMT plans using the LMT assessment template and based on the customizable assessment.

20. The one or more computer storage media of claim 19, wherein the one or more LMT plans comprise a plurality of tasks and the computer-executable instructions further cause the processor to receive feedback relating to one or more tasks of the plurality of tasks and process the received feedback to generate an alert.
